(19) United States
(12) Patent Application Publication
(10) Pub. No.: US 2011/0104780 A1
(43) Pub. Date: May 5, 2011

(54) ENCAPSULATION OF LIVING CELLS WITHIN AN AEROSOLIZED SOL-GEL MATRIX

(75) Inventors: David Benjamin Jaroch, Lafayette, IN (US); Jenna Leigh Rickus, West Lafayette, IN (US)

(73) Assignee: PURDUE RESEARCH FOUNDATION

(21) Appl. No.: 13/000,512

(22) PCT Filed: Jun. 25, 2009
(86) PCT No.: PCT/US09/48665

§ 371 Date: Dec. 21, 2010

Related U.S. Application Data
(60) Provisional application No. 61/075,587, filed on Jun. 25, 2008.

(51) Int. Cl. C12N 11/14

(52) U.S. Cl. 435/176

(57) ABSTRACT

A method of encapsulating a population of cells in a porous matrix is disclosed. The method comprises the steps of providing a silica sol mixture, aerosolizing the silica sol mixture to form a silica sol vapor, and coating the cell population with the silica sol vapor, wherein the vapor condenses to form a sol-gel matrix encapsulating the cell population.

### CROSS REFERENCE TO RELATED APPLICATIONS

This application claims priority under 35 U.S.C. §119(e) to U.S. Provisional Application Ser. No. 61/075,587 filed Jun. 25, 2008, the disclosure of which is incorporated herein by reference.

### FIELD OF THE INVENTION

The present disclosure pertains to the field of biomedical and biological engineering. More particularly, the present disclosure pertains to a method of encapsulating living cells within an aerosolized matrix.

### BACKGROUND

The integration of cells into engineered devices has considerable potential in implantable biomedical therapeutics, stem cell environments and cell-based biosensors. These applications require that cells survive on or in inorganic or hybrid materials and carry out normal metabolism. The microenvironment immediately surrounding the cells substantially impacts cellular processes and ultimately the final fate of the cell. Recent advances in technology have allowed for the integration of man-made substances with cellular materials to create a new class of living composite devices. Such devices have the ability to respond dynamically with biological functionality to their local environment.

One potential class of synthetic material for hybrid cellular applications is sol-gel derived silica glasses. Such materials are biocompatible in soft and hard tissue applications. Silica based sol-gels also possess a mesoporous architecture, allowing free diffusion of small molecules while preventing penetration of larger structures such as cells. Sol-gels can be synthesized at room temperature in aqueous environments with specialized formulations capable of generating non-cytotoxic liquid intermediate sols.

Current aerosolizing processes use heat to move liquid silica precursors into the vapor phase and then flow the vapor across partially dried cell surfaces. Such methods are limited to the use of precursors with high vapor pressures and low atmospheric boiling points. Precursors with low vapor pressures require too much heat to vaporize, and high temperature vapor streams can damage cells and degrade bioactive materials.

Current methods of coating cells with thin films are limited in the types of cells that can be encapsulated, the types of precursors that can be used, and the additional substances that may be incorporated into the aerosol, such as drugs or bioactive agents. Improvements to the method allow a much wider range of precursors to be utilized, including, for example, organically modified silanes and peptide modified precursors.

### SUMMARY OF THE INVENTION

A method for encapsulating cells in a sol-gel matrix is herein described. In one embodiment, a method of encapsulating cells is described. The method comprises the steps of providing a silica sol mixture, aerosolizing the silica sol mixture to form a silica sol vapor, and coating the cells with the silica sol vapor, wherein the vapor condenses to form a porous sol-gel matrix encapsulating the cells.

In the above described embodiment, the following features, or any combination thereof, apply. In the above described embodiment, the silica sol mixture can be aerosolized with a nebulizer, the sol-gel matrix can be a mesoporous matrix, the silica sol mixture can comprise a silicate, the silicate can be selected from the group consisting of tetraethylorthosilicate, tetramethylorthosilicate, and tetrapropylorthosilicate, the silica sol mixture can comprise a peptide, the silica sol mixture can comprise a cell population, the silica sol mixture can comprise a pharmaceutical agent, the silica sol mixture can be aerosolized at room temperature, the silica sol mixture can be aerosolized at a temperature in the range of about 18° C. to about 37° C.

In one embodiment, a method of preparing a porous sol-gel matrix is described. The method comprises the steps of providing a silica sol mixture, aerosolizing the silica sol mixture to form a silica sol vapor, coating the silica sol vapor onto a surface, wherein the silica sol vapor condenses to form a porous sol-gel matrix.

In the above described embodiment, the following features, or any combination thereof, apply. In the above described embodiment, the silica sol mixture can be aerosolized with a nebulizer, the sol-gel matrix can be a mesoporous matrix, the silica sol mixture can comprise a silicate, the silicate can be selected from the group consisting of tetraethylorthosilicate, tetramethylorthosilicate, and tetrapropylorthosilicate, the silica sol mixture can comprise a peptide, the silica sol mixture can comprise a cell population, the silica sol mixture can comprise a pharmaceutical agent, the silica sol mixture can be aerosolized at room temperature, the silica sol mixture can be aerosolized at a temperature in the range of about 18° C. to about 37° C.

In one embodiment, an apparatus for encapsulating a cell population is described. The Apparatus comprises an air pump connected by a first conduit to a vent filter, said vent filter connected via a second conduit to a nebulizer, said nebulizer connected via a third conduit to a vapor chamber wherein a cell population can be housed.

### BRIEF DESCRIPTION OF THE DRAWINGS

FIG. 1 is a view of an assembly for aerosolizing a sol-gel precursor and spraying of the aerosol onto living cells.

FIG. 2 depicts a bright field light microscopy image of P19 cells cultured on a tissue culture dish for 1 day after a 60 second sol-gel coating period.

FIG. 3 depicts a bright field light microscopy image of P19 cells cultured on a tissue culture dish for 2 days after a 30 second sol-gel coating period.

FIG. 4 is a fluorescence microscopy image of Hoechst stained P19 cellular nuclei cultured on a tissue culture dish for 1 day after a 60 second sol-gel coating period.

FIG. 5 shows confocal fluorescence images of living P19 cells at 1 day (a) and 2 days (c) post-30 second coating with sol-gel. Control samples cultured for 1 day (b) and 2 days (d) without sol-gel coating display typical cell growth in the absence of a confining layer.

FIG. 6 shows confocal fluorescence images of Live/Dead fixable dead cell stained P19 cells cultured on a tissue culture dish for 1 day after a 30 second sol-gel coating period. Positive dead cell stain (illustrated by arrow) is characterized by high intensity fluorescence.

FIG. 7 shows oxygen influx at the cellular surface 1 hour after coating cells with sol-gel vapor. Addition of metabolic disrupters chlorocarbonyl cyanide phenyl-hydrazone (CCCP) to the media resulted in increased oxygen influx.

FIG. 8 shows proton efflux at the cellular surface 48 hours after coating cells with sol-gel vapor. Addition of different metabolic disrupters both increased (CCCP, antimycin A) and decreased (NaN₃, oligomycin) efflux from the cells. Panel A: addition of CCCP, oligomycin, and NaN₃. Panel B: addition of antimycin A and NaN₃.

### DETAILED DESCRIPTION

While the invention is susceptible to various modifications and alternative forms, specific embodiments will herein be described in detail. It should be understood, however, that there is no intent to limit the invention to the particular forms described, but on the contrary, the intention is to cover all modifications, equivalents, and alternatives falling within the spirit and scope of the invention.

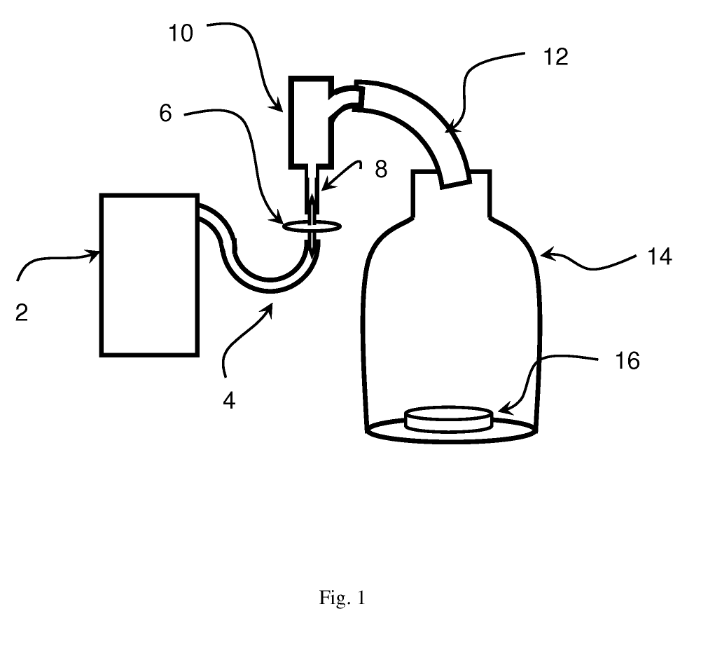

In accordance with one embodiment of the invention, an apparatus is assembled for the aerosolizing of silica-sol and spraying of the vapor onto cells. As shown in FIG. 1, the apparatus comprises an air pump 2 connected by a first conduit 4 to a vent filter 6. The vent filter 6 is connected via a second conduit 8 to a nebulizer 10, which is connected via a third conduit 12 to a vapor chamber 14. The cell population 16 to be coated by the aerosolized material sits within the vapor chamber 14. Any other suitable apparatus known in the art can be used.

In accordance with another embodiment of the invention, a method for encapsulating cells within an aerosolized sol-gel matrix is provided. The method comprises the steps of providing silica sol mixture, aerosolizing the silica sol mixture to form a silica sol vapor, coating (e.g., by spraying) the silica sol vapor onto the surface of a population of cells, and allowing the solvent to evaporate to form a porous sol-gel matrix. In one illustrative embodiment, the mixture is aerosolized with a nebulizer. The silica sol mixture is prepared, for example, by mixing a silica precursor (e.g., a silicate) with an excess of water.

In one illustrative embodiment, a nebulizer is used to aerosolize the sol-gel material into a fine mist of liquid sol particles. However, any device for aerosolizing a liquid into a fine mist or vapor may be used, e.g., a medical nebulizer, an atomizer, a vaporizer, an aerosol generator, or the like.

As used herein the term sol-gel refers to a composition formed from a solution containing metal alkoxide or metal chloride colloidal precursors (a sol), which undergo hydrolysis and polycondensation reactions to form an inorganic network containing a liquid phase (gel). The formed matrix can be subjected to a drying process to remove the liquid phase from the gel thus forming a porous material. For example, in one embodiment a sol-gel is formed from orthosilicates, including for example, tetramethylorthosilicate, tetrapropylorthosilicate, and tetraethylorthosilicate.

In various illustrative aspects, hybrid cellular materials are generated by transforming sol-gel bulk liquids into vapors. A nebulizer is used to aerosolize a specially formulated liquid sol intermediate. In various illustrative embodiments, microscopic droplets of sol are carried in a gas stream to a cell culture plate, where they wick around exposed surfaces in liquid form. The silica species in the sol can rapidly polycondense to form a mesoporous solid, encapsulating the cellular material upon which it is deposited.

In one exemplary embodiment, a silica sol mixture with an excess of water (e.g., 1:12 molar ratio of water to a silica precursor, such as tetramethyl orthosilicate (TMOS)), is catalyzed under agitation in a sonicator at room temperature using a low concentration of acid (e.g. HCl). In another embodiment, the resultant sol contains a weakly acidic mixture of methanol, water, and silicon monoxide (Si—O) groups. The solution is sonicated to facilitate hydrolysis. In another embodiment, excess methanol is removed by rotary evaporation under vacuum. Any silica precursor can be used in the silica sol mixture as herein described. For example, silica precursors can include silicates, such as tetramethylorthosilicate, tetraethylorthosilicate, and tetrapropylorthosilicate.

In various illustrative embodiments, the silica sol mixture is catalyzed using a hydrochloric acid solution, but any other acids including acetic acid, formic acid, lactic acid, citric acid, sulfuric acid, ethanoic acid, carbonic acid, nitric acid, or phosphoric acid can be used. For example, acids, at concentrations of from about 0.001 M to about 0.1 M, from about 0.04 M to about 0.1 M, from about 0.005 M to about 0.1 M, from about 0.01 M to about 0.1 M, from about 0.05 M to about 0.1 M, from about 0.001 M to about 0.05 M, from about 0.001 M to about 0.01 M, 0.01 M to about 0.04 M, or from about 0.01 M to about 0.05 M can be used as a catalyzing agent.

In various illustrative aspects, the population of cells remains metabolically active following encapsulation with the sol get matrix. It should be appreciated that cells are able to survive and maintain functionality affixed under the sol gel layer described herein. The mesoporous architecture of the sol gel matrix allows transport of nutrients into the cells. Useful cellular products, such as hormones, growth factors, neurotransmitters, or other signaling molecules, can also diffuse from the cells into surrounding tissue or material environment. In various illustrative embodiments, the sol-gel matrix can serve as a physical barrier to the immune system when implanted into a host. In various aspects, uncontrolled cell growth is restricted in sol gel encapsulated cell populations compared to control cell populations.

According to one embodiment, a silica sol vapor is generated for spray coating living cells. Any combination of suitable gasses may be used. The vapor may be generated at room temperature under normal laboratory conditions, which allows for the incorporation of cells, drugs, and biological agents that might otherwise be destroyed by high temperatures or strong sonication.

The methods herein described are amenable to being carried out under a wide range of temperatures, ranging from the freezing point to boiling point of the sol and including room or culture temperatures (e.g., from about 18° C. to about 37° C.) under normal laboratory conditions.

In one embodiment, vapor mediated deposition of the silica sol enables the coating of complex three dimensionally shaped structures, e.g. biological implants, in a controllable fashion. The structures may be coated by preparing a silica sol mixture, aerosolizing the silica sol mixture to form a silica sol vapor, and coating the silica sol vapor onto the surface of the structure, wherein the silica sol vapor condenses to form a porous sol-gel matrix. Further, complex lamellar glasses can be produced with individual layers tailored for specific functions. In another embodiment, the method disclosed herein allows for the encapsulation of a wide variety of living cells, as herein described, for use in sensors or as adaptive drug delivery devices.

In various illustrative embodiments, the sol particles are allowed to passively, or actively (e.g., by use of an electric or magnetic field), coat three dimensional surfaces, such as device surfaces (e.g., biological implants and other devices) or the surface of a population of cells. Device surfaces may contain organic or inorganic components and/or a population of cells cultured in buffered media solution. In one embodiment, evaporation of the solvent initiates the polycondensation of silica glass on dry surfaces. Residual buffered media coating the cells also acts to rapidly polycondense the silica to form a mesoporous glass.

Various agents such as nanoparticles, pharmacological agents, biomolecules, and cells can be suspended or dissolved in the sol at any time. Additionally, a number of agents or gasses may be combined. In various illustrative embodiments, a variety of pharmaceutical agents, nanoparticles, biomolecules (e.g., peptides), and cell populations, can be incorporated into the sol prior to aerosolization. Various particular agents can be added to the sol to prevent apoptosis of the encapsulated cells, locally suppress immune system responses, support tissue integration at the sol-gel interface, or to modify other activities.

In various aspects, the agents to be combined with the sol include nutrients, such as minerals, amino acids, sugars, peptides, proteins, vitamins, or glycoproteins, such as laminin and fibronectin, hyaluronic acid, anti-inflammatory agents, or growth factors such as epidermal growth factor, platelet-derived growth factor, transforming growth factor beta, or fibroblast growth factor, and glucocorticoids.

As described herein, the cell population may comprise one or more cell populations. In various illustrative embodiments, the cell population may be a eukaryotic cell population or a prokaryotic cell population, e.g., mammalian cells, yeast, or bacterial cells. In various embodiments, the cell populations comprise a population of mesodermally derived cells selected from the group consisting of endothelial cells, neural cells, blood cells, pericytes, osteoblasts, fibroblasts, endothelial cells, epithelial cells, pancreatic cells (e.g., pancreatic islet cells, pancreatic beta cells, etc.), smooth muscle cells, skeletal muscle cells, cardiac muscle cells, mesenchymal cells, adipocytes, adipose stromal cells, stem cells (e.g., totipotent, multipotent, and pluripotent stem cells), osteogenic cells, or combinations thereof.

As used herein, stem cells refer to an unspecialized cell from an embryo, fetus, or adult that is capable of self-replication or self-renewal and can develop into specialized cell types of a variety of tissues and organs (i.e., potency). The term as used herein, unless further specified, encompasses totipotent cells (those cells having the capacity to differentiate into extra-embryonic membranes and tissues, the embryo, and all post-embryonic tissues and organs), pluripotent cells (those cells that can differentiate into cells derived from any of the three germ layers), multipotent cells (those cells having the capacity to differentiate into a limited range of differentiated cell types, e.g., mesenchymal stem cells, adipose-derived stem cells, endothelial stem cells, etc.), oligopotent cells (those cells that can differentiate into only a few cell types, e.g., lymphoid or myeloid stem cells), and unipotent cells (those cells that can differentiate into only one cell type, e.g., muscle stem cells). Stem cells may be isolated from, for example, circulating blood, umbilical cord blood, or bone marrow by methods well-known to those skilled in the art.

In various illustrative embodiments, the peptides incorporated into the sol may be naturally occurring amino acids or synthetic (non-naturally occurring) amino acids or a mixture of naturally occurring and synthetic amino acids. Synthetic or non-naturally occurring amino acids refer to amino acids that do not naturally occur in vivo but which, nevertheless, can be incorporated into the peptide structures described herein. A general reference to a “peptide” or “amino acid” is intended to encompass the possible inclusion of synthetic or non-naturally occurring amino acids. In addition, the present disclosure also encompasses the possible further modification of the peptides to include additional biochemical functional groups such as acetate, phosphate, lipid and carbohydrate moieties. In one embodiment, the peptides incorporated into the sol are peptide-silane compounds as described in International Patent Application Number PCT/US2007/081122, incorporated herein by reference.

The methods as herein described allow for the encapsulation of living cells, both prokaryotic and eukaryotic, for use in sensors or as adaptive drug delivery devices. For example, the methods herein described allow for the implantation of foreign cellular material into a host without the need for global suppression of the immune system of the host.

### EXAMPLES

### Example 1

Embryonic carcinoma derived stem cells (P19 cell line) were immobilized in a thin film of unmodified silica using the above described vaporized sol-gel technique. The cells survive and are metabolically active in the materials. Uncontrolled cell growth is restricted compared to controls.

### Sol-Gel Synthesis

Saturated silica sol was formed by the acid-catalyzed hydrolysis of tetramethyl orthosilicate (TMOS). TMOS and deionized H₂O (DiH₂O) were combined at a 1 to 12 mol ratio. A small amount of 0.04 M HCl solution (2 μl per 1 gram of TMOS/H₂O solution) was added as the catalyzing agent. The solution was sonicated for 15 minutes until the completion of hydrolysis (characterized by clear homogonous sol formation). Excess methanol was then removed by rotary evaporation under vacuum (35° C. water bath, 5 min evaporation time).

### Glass Slide Preparation

Organic residue was removed from the surface of 8 mm diameter glass cover slip discs using piranha solution (3 parts H₂SO₄, 1 part 30% H₂O₂ solution, 3 hour soak time). The slides were then rinsed in DiH₂O and placed in a centrifuge tube with ethanol and DiH₂O to maintain sterility. Prior to cell culture, the cover slips were placed into a non tissue culture treated Petri dish under sterile conditions. The surfaces of the glass discs were then covered in approximately 20 μl of poly-L-lysine solution for 20 minutes to facilitate cell attachment. Excess poly-L-lysine was removed by subsequent rinsing with 5 ml of cell culture media.

### Cell Culture

Pluripotent murine P19 embryonic carcinoma cells were grown to 80% confluence, dissociated, centrifuged to form a pellet, and resuspended in 10 ml of media. A 1 ml aliquot of suspended cells was then added to 9 ml of media in the tissue culture plate containing the glass slides. The cells were then allowed to adhere to the discs over a 24 hour incubation period. Additional samples were prepared by adding 1 ml of suspended cells to 9 ml of media in tissue culture treated Petri dishes. These samples were also allowed to incubate for 24 hours to facilitate attachment.

### Sol Gel Coating

A Pari LC Plus medicinal nebulizer, 0.2 μm air line filter, and associated air line tubing were autoclaved prior to coating. Sol-gel was then filtered twice through 0.2 μm syringe filters and introduced to the medicine cup of the nebulizer. Media was removed from the cell culture dishes. The samples were then placed under a vapor chamber constructed from a 500 ml plastic Nalgene sample bottle with removed bottom. The nebulizer pump was then activated and the resulting sol-gel vapor was introduced to the sample chamber for 30 or 60 seconds. The chamber was removed immediately after the coating period. The samples were allowed to rest for 20 seconds post coating to allow for the polycondensation of the sol into a solid gel. After the resting period, 10 ml of media was then introduced to the samples, which were then allowed to incubate for 24 or 48 hours.

### Cell Staining and Fixation

### Live/Dead Fixable Dead Cell/Hoechst Co-Stain

After incubation, the coated cells were washed twice with PBS. Reconstituted fluorescent reactive dye solution (1 μl dye to 1 ml PBS) was introduced to the plates followed by incubation at room temperature for 30 minutes. The samples were then washed 3 times with PBS to remove residual stain. After staining with fixable dead cell stain, the samples were fixed using a 3.7% formaldehyde/PBS solution, followed by a 15 minute incubation period at room temperature. The samples were then washed twice to remove residual formaldehyde. Hoechst DNA staining was performed after fixation by introducing 2 μl concentrated 10 mg/mL Hoechst stock solution per ml of PBS in the culture plate. Samples were wrapped in tin foil to prevent photobleaching and stored into a refrigerator at 4° C. prior to analysis.

### Mitotracker/Hoechst Co-Stain

MitoTracker stock solution was diluted to 1 mM concentration in DMSO. Working solution was then prepared by the addition of 4 μl per 10 ml of cell culture media to obtain a 400 nM staining solution. The media was then removed from the sample dish and 3 ml of pre-warmed (37° C.) growth media containing the MitoTracker probe was added. The plates were then allowed to incubate for 45 minutes. After staining, the samples were washed in fresh, pre-warmed growth media. The cells were then fixed using pre-warmed growth medium containing 3.7% formaldehyde followed by incubation at 37° C. for 15 minutes. After fixation, the cells were rinsed several times in PBS. Hoechst DNA staining was performed after fixation by introducing 2 μl concentrated 10 mg/mL Hoechst stock solution per ml of PBS in the culture plate. Samples were then wrapped in tin foil to prevent photobleaching and stored into a refrigerator at 4° C. prior to analysis.

### Microscopy

Images of the samples were collected using bright field light microscopy. Fluorescence microscopy was also performed in order to obtain images of blue Hoechst stained cellular nuclei. Confocal microscopy was conducted to determine if living cells (characterized by a blue Hoechst stained nucleus surrounded by red MitoTracker stained mitochondria) were present after coating with sol-gel. Characterization of dead cell populations (blue Hoechst stained nucleus surrounded by red Live/Dead stained cellular material) was also performed.

### Bright Field Light Microscopy

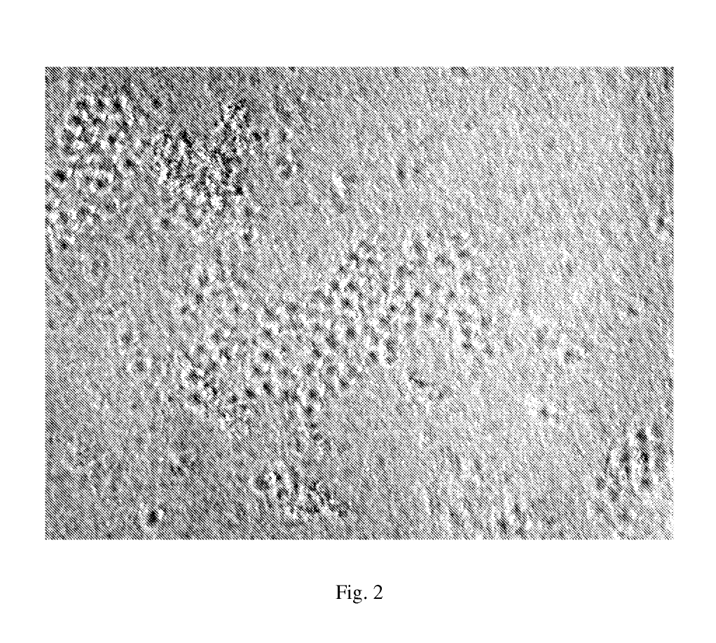

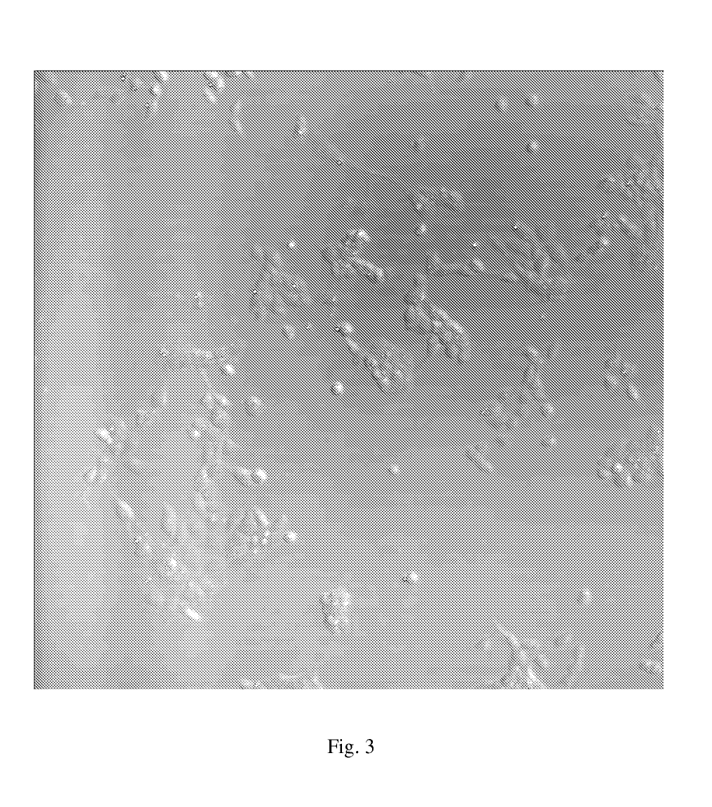

Bright field light microscopy revealed clear populations of healthy looking cells (FIG. 2). Close examination of both acellular and cell covered regions of the Petri dish demonstrated a rough, pebble-like texture associated with the polycondensation of nebulized sol-gel droplets contacting the cell and plate surface. The presence of this layer over both the cells and Petri dish indicate that uniform coverage and encapsulation were obtained. Images taken 2 days post-coating clearly display healthy looking cells at concentrations similar to the original plating density at the time of coating (FIG. 3). Textural features arising from the sol-gel coating are not as apparent due to a shorter coating time.

### Fluorescence Microscopy

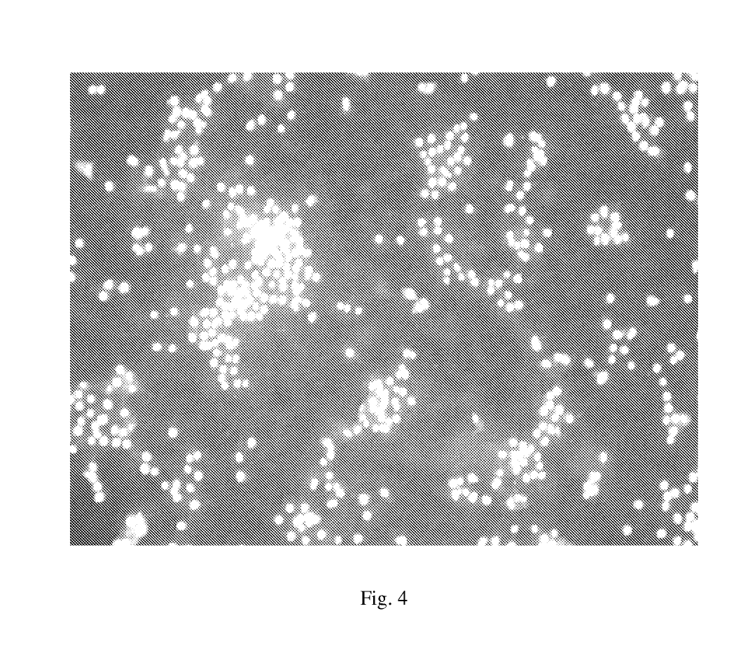

Cellular material was confirmed by Hoechst nuclear staining under fluorescence microscopy (FIG. 4). Coherent bright blue nuclei are clearly present throughout the sample, indicating that the sol gel successfully entrapped the cells.

### Confocal Microscopy

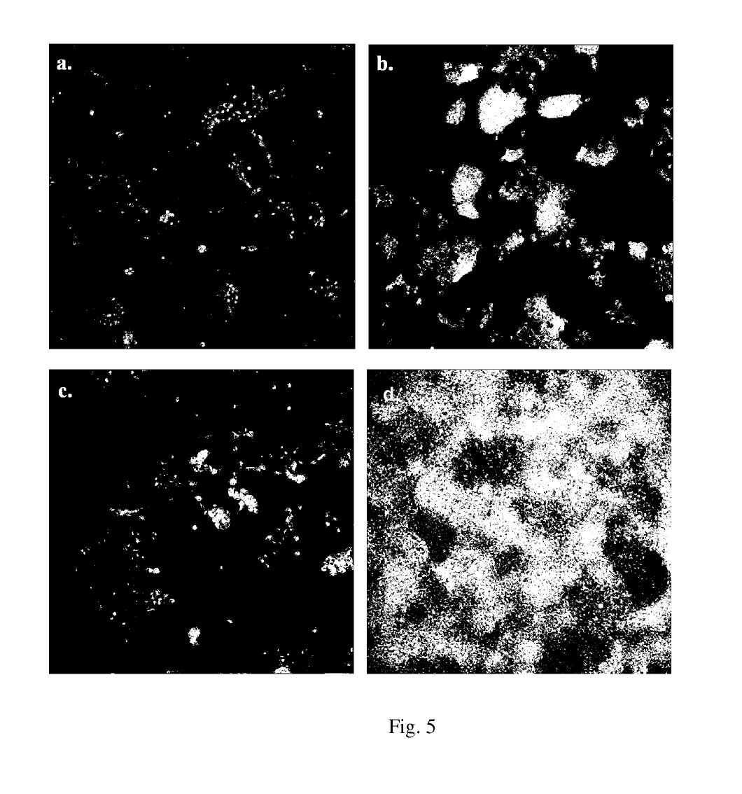

Live cell staining for active mitochondria (MitoTracker) demonstrated healthy populations of cells 1 day (FIG. 5a) and 2 days (FIG. 5c) after 30 seconds of coating with the sol-gel. Control samples cultured for 1 day (FIG. 5b) and 2 days (FIG. 5d) without sol-gel coating display typical cell growth in the absence of a confining layer. The effects of cellular entrapment are illustrated by comparison with the growth pattern of cells allowed to incubate for 24 and 48 hours without a sol-gel coating (FIG. 5b & d, respectively). Live cell staining was conducted using MitoTracker and Hoechst stains.

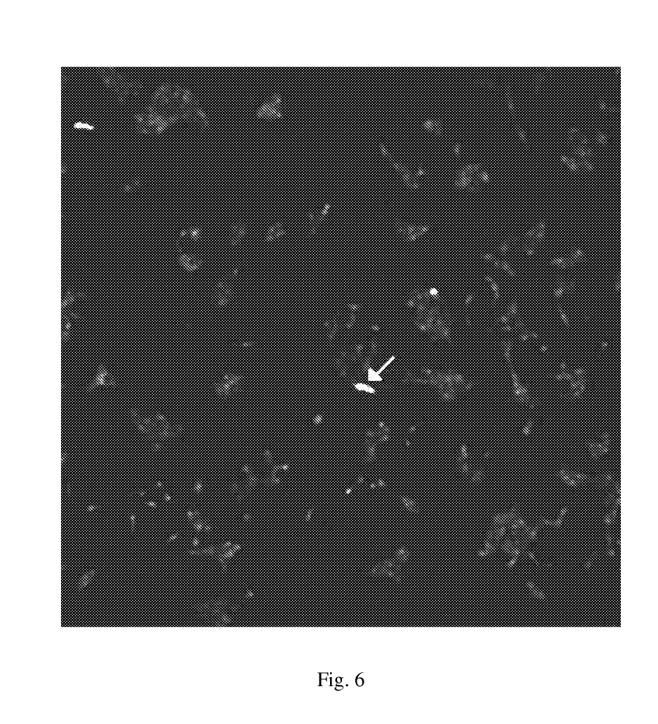

Live/Dead fixable dead cell staining was utilized to determine if the sol gel coating induced a cytotoxic response (FIG. 6). Dead cells stain bright red and bind a larger quantity of the fluorescent dye, increasing their subsequent fluorescent intensity upon analysis. A relatively small number of positively stained dead cells were observed relative to the live cell population.

The results of this study indicate that P19 cells can survive the initial deposition of sol-gel vapor. Gross physical examination of the cell bodies under light microscopy provides visual evidence of an apparently healthy cell population (FIG. 3). Sufficient nutrient transfer took place to allow for critical metabolic activity, as indicated by active mitochondria (positive MitoTracker staining), over at least a two-day incubation period (FIG. 5). Few dead cells were apparent after staining with the Live/Dead dead fixable stain (FIG. 6). These results cumulatively suggest that the presently disclosed deposition technology induces minimal cytotoxic effects upon application, and that the porous structure of the solid gel is capable of allowing for the diffusion of molecules necessary for cell viability over time.

Visual confirmation of sol-gel layer formation was made on samples coated for 60 seconds under bright field light microscopy (FIG. 2). Functional confirmation of a sol-gel layer can be made by observing the growth pattern of samples lacking a sol-gel coating (FIG. 5b & c). The cell density of such samples was much higher than that of sol-gel coated samples incubated for the same length of time (FIG. 5a & c, respectively). Sol-gel coated samples maintained a cell density similar to that observed immediately prior to vapor deposition. Once encased in the sol-gel layer, cells are physically confined and cannot divide and spread into the large confluent populations observed in the sol-free controls. The ability of the material to physically confine rapidly dividing cells and reduce uncontrolled cell growth may reduce the risk of transplanted cells forming tumors after transplantation into a host.

### Example 2

Data relating to oxygen influx and proton efflux were collected using a self referencing electrode apparatus.

### Oxygen Influx

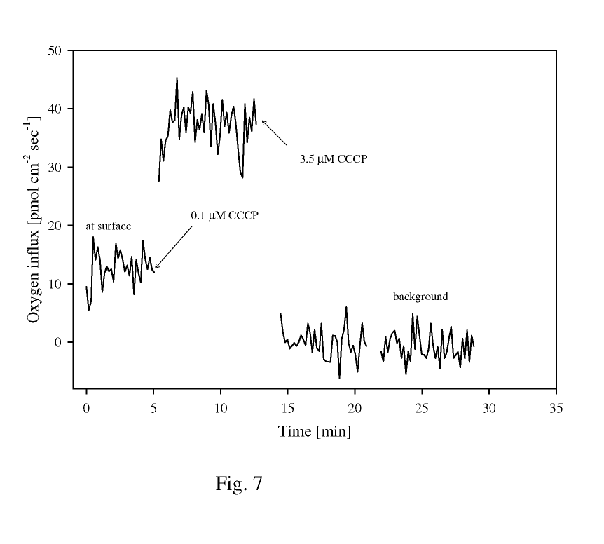

Oxygen influx measurements at the coating surface demonstrated the active intake of oxygen, indicating that the coated cells were metabolically active. Oxygen influx at the cellular surface 1 hour after coating with sol-gel vapor is shown in FIG. 7. The addition of chlorocarbonyl cyanide phenyl-hydrazone (CCCP), a metabolic disrupter, generated increased influx of oxygen. This data demonstrates that the cells are metabolically active and can respond to pharmacological stimuli after encapsulation.

### Proton Efflux

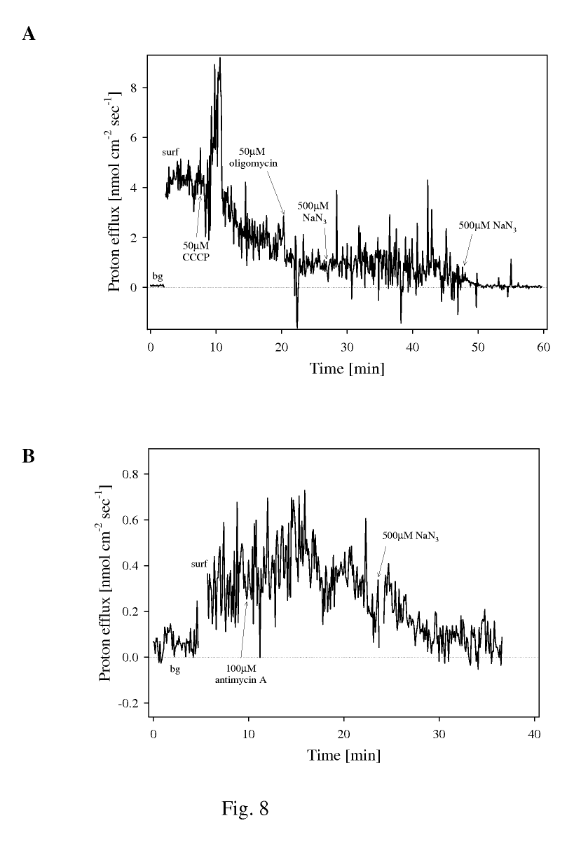

Proton efflux is the result of a variety of cellular properties including metabolism. Proton efflux was detected at the cellular surface 48 hours after coating cells with the sol-gel vapor, indicating that the cells were alive and active (FIG. 8, Panels A and B). The addition of a variety of metabolic disrupters influenced proton efflux with CCCP and antimycin A increasing efflux, and oligomycin and NaN₃ decreasing efflux.

### Example 3

### Sol Gel Coating of Bacterial Cells

Sol gel vapor coating has been used to immobilize bacterial cells (data not shown). For example, experiments have been conducted to coat cellular surfaces to encapsulate and immobilize Escherichia coli and Pseudomonas bacteria. The technique is not limited to these strain and can be applied to a wide variety of bacteria.

While the invention has been illustrated and described in detail in the foregoing description, such an illustration and description is to be considered as exemplary and not restrictive in character, it being understood that only the illustrative embodiments have been described and that all changes and modifications that come within the spirit of the invention are desired to be protected. Those of ordinary skill in the art may readily devise their own implementations that incorporate one or more of the features described herein, and thus fall within the spirit and scope of the present invention.

What is claimed is:

1. A method of encapsulating a cell population, said method comprising the steps of,
providing a silica sol mixture,
aerosolizing the silica sol mixture to form a silica sol vapor; and
coating the cell population with the silica sol vapor, wherein the vapor condenses to form a sol-gel matrix encapsulating the cells.

2. The method of claim 1 wherein the silica sol mixture is aerosolized with a nebulizer.

3. The method of claim 1 wherein the sol-gel matrix is a mesoporous matrix.

4. The method of claim 1 wherein the silica sol mixture comprises a silicate.

5. The method of claim 4 wherein the silicate is selected from the group consisting of tetraethylorthosilicate, tetramethylorthosilicate, and tetrapropylorthosilicate.

6. The method of claim 1 wherein the silica sol mixture comprises a peptide.

7. The method of claim 1 wherein the silica sol mixture comprises a cell population.

8. The method of claim 1 wherein the silica sol mixture comprises a pharmaceutical agent.

9. The method of claim 1 wherein the silica sol mixture is aerosolized at room temperature.

10. The method of claim 1 wherein the silica sol mixture is aerosolized at a temperature in the range of about 18° C. to about 37° C.

11. A method of preparing a porous sol-gel matrix, said method comprising the steps of,
providing a silica sol mixture,
aerosolizing the silica sol mixture to form a silica sol vapor;
coating the silica sol vapor onto a surface, wherein the silica sol vapor condenses to form a porous sol-gel matrix.

12. The method of claim 11 wherein the silica sol mixture is aerosolized with a nebulizer.

13. The method of claim 11 wherein the porous sol-gel matrix is a mesoporous matrix.

14. The method of claim 11 wherein the silica sol mixture comprises a silicate.

15. The method of claim 14 wherein the silicate is selected from the group consisting of tetraethylorthosilicate, tetramethylorthosilicate, and tetrapropylorthosilicate.

16. The method of claim 11 wherein the silica sol mixture comprises a peptide.

17. The method of claim 11 wherein the silica sol mixture comprises a population of cells.

18. The method of claim 11 wherein the silica sol mixture comprises a pharmaceutical agent.

19. The method of claim 11 wherein the silica sol mixture is aerosolized at room temperature.

20. The method of claim 11 wherein the silica sol mixture is aerosolized at a temperature in the range of about 18° C. to about 37° C.
